(19) United States
(12) Patent Application Publication
(10) Pub. No.: US 2015/0166441 A1
(43) Pub. Date: Jun. 18, 2015

(54) METHODS AND SYSTEMS FOR REMOVING ACETONE FROM OLEFIN-CONTAINING HYDROCARBON STREAMS

(71) Applicant: UOP LLC, Des Plaines, IL (US)

(72) Inventor: Kurt A. Detrick, Glen Ellyn, IL (US)

(73) Assignee: UOP LLC, Des Plaines, IL (US)

(21) Appl. No.: 14/104,759

(22) Filed: Dec. 12, 2013

(51) Int. Cl. C07C 7/148

(52) U.S. Cl. CPC C07C 7/148

(57) ABSTRACT

Disclosed are methods and systems for removing acetone from olefin-containing hydrocarbon streams. In one embodiment, a method for removing acetone from a hydrocarbon stream includes mixing an olefin-containing hydrocarbon stream and a wash water stream to form a mixed olefin/wash water stream, wherein the olefin-containing hydrocarbon stream comprises acetone impurities, and wherein mixing comprises dissolving at least a portion of the acetone impurities in to the wash water stream, coalescing the mixed olefin/water stream to separate the wash water stream with acetone dissolved therein from the hydrocarbon stream, wherein the separated hydrocarbon stream comprises non-coalesced wash water and acetone, and drying the separated hydrocarbon stream to remove at least a portion of the non-coalesced wash water and acetone therefrom and to form a dried hydrocarbon product stream.

### TECHNICAL FIELD

The present disclosure generally relates to methods and systems for treating hydrocarbon streams. More particularly, the present disclosure relates to methods and systems for removing acetone from olefin-containing hydrocarbon streams.

### BACKGROUND

The fluid catalytic cracking (FCC) process is a process for the conversion of straight-run atmospheric gas oil, vacuum gas oils, certain atmospheric residues, and heavy stocks recovered from other operations into high-octane gasoline, light fuel oils, and olefin-rich light gases. In a petroleum refinery the FCC unit typically processes about 30 to about 50 percent of the crude oil charged to the refinery. Early FCC units were designed to operate on vacuum gas oils directly fractionated from crude oils. Typically, these vacuum gas oils came from highly quality crude oils. Today, much of the high quality feedstock for FCC units has been depleted and modern FCC units process less favorable materials. These less favorable materials include a substantial amount of sulfur containing materials and a growing portion of the non-distillable fraction of the crude oil. As a result, the contaminant level of the FCC product fractions have increased, particularly in the C₃ -C₅ product fraction. Without appropriate treatment, the contaminants in the C₃ -C₅ product fractions can be transmitted to sensitive downstream processes where they reduce the effectiveness of downstream catalysts and create unfavorable by-product reactions in processes such as alkylation.

Alkylation reactions are typically carried out in a liquid phase in the presence of a concentrated HF or H₂SO₄ acid catalyst in a reaction zone. From the reaction zone, the hydrocarbon products and the catalyst are separated, and the catalyst phase is returned to the reaction zone. The hydrocarbon products are fractionated to produce propane, recycle isobutane, normal butane and alkylate. In a typical HF alkylation unit with an external acid regenerator, a portion of the catalyst phase is withdrawn as a drag stream and charged to the acid regenerator. The acid regenerator separates acid soluble oils formed in the reaction zone, and an azeotrope of HF acid and water from the drag stream. The regenerated HF acid is cooled and returned to the reactor. The presence of water in the feed results in a loss of acid by the formation of the HF acid/water azeotrope. The presence of other impurities such as sulfur lead to the formation of acid soluble oils.

Another problematic impurity is acetone. Acetone levels in the olefin feedstock to alkylation units have been increasing over the past several years due to operating conditions in the upstream FCC units. The higher levels of acetone are making it difficult to keep high enough acid purity in the alkylation units to maintain good operation. In many cases, the higher acetone levels have caused significantly higher acid losses. In addition to alkylation units, it has been observed that indirect alkylation processes (such as the InAlk™ process available from UOP LLC of Des Plaines, Ill., USA) may also benefit from acetone removal.

One conventional process for removing water and other contaminants such as acetone from light hydrocarbons employs a bed arrangement that contains a molecular sieve as an absorbent material. A light hydrocarbon stream is passed through the bed arrangement and the molecular sieve absorbs much of the water and other contaminants from the stream to produce a clean hydrocarbon product. As the molecular sieve removes the undesirable components from the stream, its surface and pores become saturated with water and to a lesser extent the other contaminants, causing the molecular sieve to become less active. To restore its activity, the molecular sieve is regenerated at higher temperatures to help remove the absorbed water and other contaminants. Although water is readily removed from the molecular sieve during regeneration, the other contaminants tend to remain and react at the higher temperatures to form a gummy residue. The gummy residue steadily builds up during each additional regeneration, plugging the pores and causing premature permanent deactivation of the molecular sieve. Since regeneration at this point is no longer effective to restore activity, the molecular sieve needs to be replaced, which is expensive and time consuming Another problem that contributes to cost increases in such systems is that the dried feed cannot be used as regenerant, and thus another regenerant source is required.

Accordingly, it is desirable to provide economically viable, and simple to operate processes and system to enhance the octane rating of isomerized paraffins. Additionally, it would be desirable to reduce the capital and operating costs of such processes and systems. Furthermore, other desirable features and characteristics of the present disclosure will become apparent from the subsequent detailed description and the appended claims, taken in conjunction with the drawings and this background.

### BRIEF SUMMARY

The present disclosure generally provides methods and systems for removing acetone from olefin-containing hydrocarbon streams. In one exemplary embodiment, disclosed is a method for removing acetone from a hydrocarbon stream that includes mixing an olefin-containing hydrocarbon stream and a wash water stream to form a mixed olefin/wash water stream, wherein the olefin-containing hydrocarbon stream comprises acetone impurities, and wherein mixing comprises dissolving at least a portion of the acetone impurities in to the wash water stream, coalescing the mixed olefin/water stream to separate the wash water stream with acetone dissolved therein from the hydrocarbon stream, wherein the separated hydrocarbon stream comprises non-coalesced wash water and acetone, and drying the separated hydrocarbon stream to remove at least a portion of the remaining non-coalesced wash water and acetone therefrom and to form a dried hydrocarbon product stream. The method may further include recycling a portion of the dried hydrocarbon product stream to form a product regeneration stream and mixing the product regeneration stream with at least a portion of the olefin-containing hydrocarbon stream and the wash water stream

In another exemplary embodiment, disclosed is a system for a mixing unit that mixes an olefin-containing hydrocarbon stream and a wash water stream to form a mixed olefin/wash water stream, wherein the olefin-containing hydrocarbon stream comprises acetone impurities, and wherein mixing comprises dissolving at least a portion of the acetone impurities in to the wash water stream, a coalescer unit that coalesces the mixed olefin/water stream to separate the wash water stream with acetone dissolved therein from the hydrocarbon stream, wherein the separated hydrocarbon stream comprises non-coalesced wash water and acetone, and a dehydration unit that dries the separated hydrocarbon stream to remove at least a portion of the remaining non-coalesced wash water and acetone therefrom and to form a dried hydrocarbon product stream.

### BRIEF DESCRIPTION OF THE DRAWINGS

The present embodiments will hereinafter be described in conjunction with the following drawing FIGURES, wherein like numerals denote like elements, and wherein:

FIG. 1 is a process flow diagram illustrating a method implemented on a acetone removal system in accordance with various embodiments of the present disclosure.

### DETAILED DESCRIPTION

The following detailed description is merely exemplary in nature and is not intended to limit the application and uses of the embodiments described. Furthermore, there is no intention to be bound by any theory presented in the preceding background or the following detailed description.

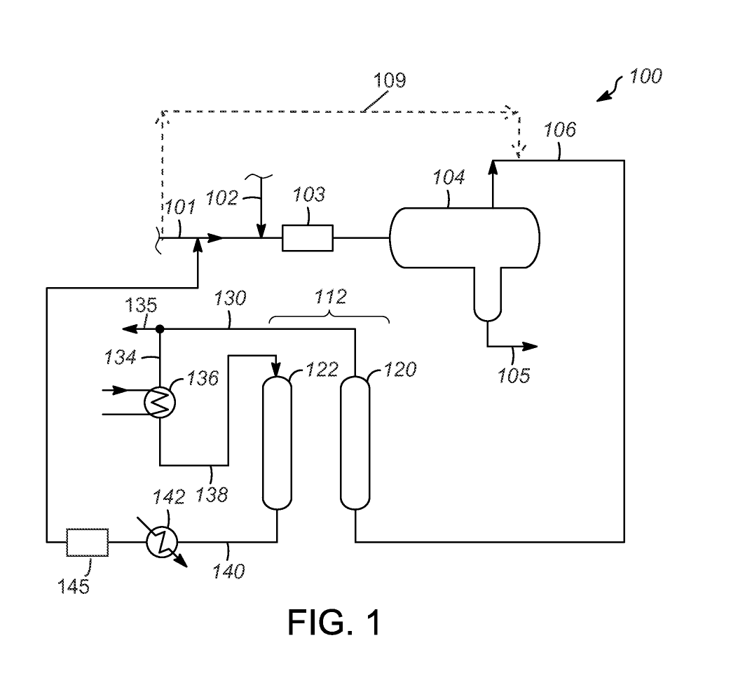

The present disclosure provides methods and systems for removing acetone from olefin-containing hydrocarbon streams. The embodiments described herein employ a novel combination of a water wash and molecular sieve adsorbers that are used to remove acetone from an olefinic feedstock more efficiently than either a water wash or molecular sieve adsorbers alone. The use of a water wash upstream of the molecular sieve adsorbers allows the use of the dried olefin feed as regenerant and the spent regenerant can be recirculated upstream of the water wash. The use of a water wash together with molecular sieve adsorbers can allow for the use of less water overall, such as less than the current typical acetone wash rates. In the practice of the described embodiments, water is added to the olefin feed and the mixture passes through an in-line mixer where a portion of the acetone dissolves into the water phase. The mixture is separated in a coalescer where the water is removed and sent downstream or to wastewater treating. The hydrocarbon phase from the coalescer is sent to a molecular sieve adsorber where the dissolved water and remaining dissolved acetone is removed. The dried effluent from the on-line first dryer unit (“Dryer A”) is then fed to the Alkylation unit reactor(s). A slipstream of the dried effluent from Dryer A is heated and used as regenerant for the off-line second dryer unit (“Dryer B”). The spent regenerant from Dryer B is cooled and returned to be blended with the olefin feed upstream of the water wash. The acetone in the spent regenerant can then be extracted in the water wash. This flow scheme will cause the acetone concentration in the combined feed (spent regenerant plus fresh olefin) to the water wash to increase or “cycle-up” until the amount of acetone that partitions into the water phase is equal to the amount of acetone in the fresh olefin feed. The embodiments are described in greater detail below with regard to FIG. 1.

FIG. 1 is a process flow diagram illustrating a method implemented on a acetone removal system 100 in accordance with various embodiments of the present disclosure. As shown in FIG. 1, a hydrocarbon feed stream is provided to system 100 via feed line 101. The hydrocarbon feedstream being treated in accordance with this disclosure may be derived from a fluid catalytic cracking (FCC) unit, a coker unit, or any other olefinic LPG feedstock source, and typically is composed of any proportion of monoolefin and paraffin, each containing from 3 to 5 carbon atoms (C₃-C₅). The paraffins include isobutane, isopentane, normal pentane, as well as propane and n-butane. The monoolefins include butene-1, butene-2, isobutene, 2-methyl-2-butene, 2-methyl-1-butene, 3-methyl-1-butene, 1-pentene, 2-pentene, cyclopentene and propylene. The hydrocarbon feedstream may also contain diolefins such as 1,3-butadiene and 1,3-pentadiene. Minor proportions of both paraffinic and olefinic molecules of various numbers of carbon atoms which can result from distillation procedures to obtain the C₃-C₅ hydrocarbons are not harmful to the process and can be present. The hydrocarbon feedstream typically contains 30 to 60 mol % olefins.

Water and its precursors may also be present in the hydrocarbon feedstream in amounts from 5 wt. ppm to saturation which typically is about 1000 wt. ppm, measured as H₂O. The contaminants may also be oxygenated hydrocarbon compounds, otherwise known as oxygenates, such as alcohols, ethers, aldehydes, ketones, and acids. Specific examples of these oxygenates are ethanol, methanol, isopropanol, tertiary butyl alcohol, dimethyl ether, methyl tertiary butyl ether, acetone, and acetic acid. Acetone may be present in trace amounts ranging from about 1 to about 1500 wt. ppm. Nitrogen compounds, particularly acetonitrile, may be present in trace amounts ranging from about 1 to about 1000 wt. ppm and more typically from about 15 to about 80 wt. ppm. Other polar compounds such as propionitrile also may be present. The feedstream may or may not have been subject to a selective hydrogenation process for the saturation of diolefins prior to its use in the pretreating process of the instant invention. Typically, the feedstream from the FCC may contain from about 1000 ppm-wt. to about 2 vol. % butadiene or diolefin. The effluent from a selective hydrogenation process will typically contain less than 50 ppm-wt. diolefins.

The hydrocarbon feed stream 101 may be provided to the system 100 in accordance with the following general process parameters. In general, the temperature will be at about 100° F. (about 38° C.). Pressures may be from about 100 to about 300 psig. Space velocity may be quite high, for example, up to 50 WHSV (hr 1) but more usually in the range of 5 to 30 WHSV. Appropriate adjustment of the process conditions will be appreciated by those having ordinary skill in the art, and as such the above-noted process conditions are not intended to be limiting.

The hydrocarbon (light olefin) feed 101 is first subjected to a wash with an aqueous wash liquid which is typically plain water, depending on the contaminants known or suspected to be present. Water is provided to system 100 via water stream 102. Water is effective for acetone and other water-soluble species such as alcohols or acetonitrile. The ratio of water to olefin is typically in the range of 0.1:1 to 10:1 by weight. As initially noted above, the presently described embodiments allow for the use of less water as compared to prior art systems, and as such water is preferable provided at 0.01:1 to 0.3:1.

The hydrocarbons from stream 101 and the wash water from stream 102 are brought together in a wash unit or “mixer” 103. Wash unit 103 design will be conventional with the objective of ensuring good contact between the olefin feed and the water with various types of contactors applicable, for example, in-line mixers, scrubbers, countercurrent towers. During this step and the following coalescence step, the conditions should be chosen so as to maintain the olefin stream in the liquid phase since this will favor removal of the contaminant species. The preferred temperatures for the feed and the wash water below about 50° C. and preferably below 40° C., since water tends to be more soluble in the feed at higher temperatures and so tends to dissolve in the feed and be less amenable to separation by coalescence and, remaining in the feed will take with it the water soluble contaminants such as acetone, alcohols, acetonitrile and nitrogenous bases. Thus, operation of the water wash and the subsequent coalescence not only reduces the water content of the feed, in itself desirable, but also reduces the level of impurities in the feed. At the preferred temperatures, the olefins will be in the liquid phase at pressures of about 100 to about 300 psig. As shown in FIG. 1, a portion of feed 101 (prior to the rejoinder of regenerant line 140, as will be described in greater detail below) may be bypassed around the wash unit 103 (shown by dashed line 109) to reduce the amount of water consumed in the washing process.

Following the water wash and mixing in unit 103 the hydrocarbon feed is separated from the water by means of a coalescer separator 104. The method of coalescing a liquid suspended in another immiscible phase using a coalescer, has been found useful for removing liquids both from the gaseous phase as in aerosols and from suspensions of one liquid in another liquid with which it is immiscible but may be soluble to a limited degree. The coalescence separation technique has become commercially attractive in recent years for separating immiscible liquids especially hydrocarbons and water. See, for example, Refining Details: Advances in Liquid/Liquid Coalescing Technology, Gardner, Today's Refinery, March 1997. Coalescing devices are particularly effective where the volume of liquid to be removed is small in comparison to the volume of the phase from which it is removed. The Gardener article discusses the factors that are relevant to the coalescence of droplets of the discontinuous phase from the continuous phase and the ease or difficulty of separation of the immiscible phases. These factors include the physical properties of the phases such as density, viscosity, surface tension and interfacial tension. In addition, the properties of the system such as drop size, curvature of the liquid/liquid interface, temperature, concentration gradients and vibrations may also affect the effectiveness of the coalescence. As noted in U.S. Pat. No. 5, 443,724 (Williamson), any or all of these factors may be significant in a particular situation but the density, drop size and interfacial tension of the two liquids appear to be the most significant factors as well as those over the least amount of control can be exercised in affecting the separations.

Prefilters that are recommended for use ahead of the coalescer may be any suitable type of conventional filter, including sand filters, metal or polymer meshes, or other porous material capable of removing small solid particles which would tend to stabilize hydrocarbon/water emulsions and which might result in damage to the more delicate coalescer membranes. Polyester and nylon mesh filters are suitable, typically with crush strengths in the range of 70-145 kg. cm² (75-150 psi) and other non-woven filter materials may be used as convenient alternatives. The filter material may be contained in a conventional filter housing and the filter material in any convenient configuration which provides the desired filter life, filtration capacity and flow rate, for example, pleated mats, cylindrical sheets or mats, helical or spirally wound mats.

In a similar manner, the material of the coalescer and separation elements in the coalescing unit and the separation unit may be provided in a form which provides the necessary mechanical strength, liquid flow rate and unit life. In the simplest form, the media serving as the coalescer and separator materials may be provided in sheet form which may be formed either as flat sheets, pleated or corrugated sheets or in other suitable arrangements e.g. cylindrically, helically or spirally wound sheets, as disclosed in U.S. Pat. No. 5,443,724 to which reference is made for a disclosure of suitable coalescer and separator materials and configurations for them. Coalescers should be selected for capability of operation at the pressures preferred for the water wash (100-300 psig).

As shown in FIG. 1, spent wash water leaves the coalescer separator via line 105. As noted above, spent wash water may be recycled, or alternatively, where an alkylation system is provided downstream of the presently described system 100, the spent water may be provided to an alkylate wash tower of the downstream alkylation system (not illustrated). As acetone is soluble in water, the spent wash water includes acetone dissolved therein, thus reducing the amount of acetone in the hydrocarbon stream. The hydrocarbon stream exits coalescer 104 via line 106. Due to contacting with the water, the hydrocarbon stream in line 106 is saturated with dissolved water, for example about 500 wt. ppm of water. As such, line 106 continues downstream to a series of dryer units included with in a regenerative dehydration zone 112, described in greater detail below.

The regenerative dehydration zone 112 includes a first dryer 120 and a second dryer 122 that work together in a swing bed arrangement. Each of the dryers contains an adsorbent such as a molecular sieve, alumina, or silica gel, that will remove water, acetone, and other contaminants from the hydrocarbon feed. As used herein, the term “molecular sieve” is defined as a class of adsorptive desiccants that are highly crystalline in nature, distinct from amorphous materials such as gamma-alumina As used herein, the terms “absorb,” “absorbed,” “absorbing,” “absorptive,” and “absorption” are used broadly and are to be understood to also include “adsorb,” “adsorbed,” “adsorbing,” “adsorptive,” and/or “adsorption.” Various types of molecular sieves include aluminosilicate materials commonly known as zeolites. As used herein, the term “zeolite” in general refers to a group of naturally occurring and synthetic hydrated metal aluminosilicates, many of which are crystalline in structure. There are, however, significant differences between the various synthetic and natural materials, such as differences in chemical composition, crystal structure and physical properties. The zeolites occur as agglomerates of fine crystals or are synthesized as fine powders and are preferably tableted or pelletized for large-scale adsorption uses.

As illustrated, the regenerative dehydration zone 112 is configured for “recirculated regenerant operation.” The first and second dryers 120 and 122 are configured as a swing bed arrangement in which one of the first and second dryers 120 and 122 is in a dehydration mode and the other of the first and second dryers 120 and 122 is in a regenerative mode. In particular, when a first plurality of valves are in an opened position and a second plurality of valves are in a closed position, the first dryer 120 is in the dehydration mode and the second dryer 122 is in the regenerative mode. Alternatively, when the first plurality of valves are in the closed position and the second plurality of valves are in the opened position, the first dryer 120 is in the regenerative mode and the second dryer 122 is in the dehydration mode.

As illustrated, the first dryer 120 in the dehydration mode receives the hydrocarbon-containing feed stream 106 and is operating at dehydration conditions. In an exemplary embodiment, the dehydration conditions include a temperature of from about 0 to about 60° C., preferably of about 0 to about 50° C., and more preferably of about ambient, and a pressure so as to maintain a liquid phase, such as, for example, of from about 350 to about 4200 kPa. The hydrocarbon-containing feed stream 106 contacts the adsorbent or dessicant and water, acetone, and other contaminants are selectively absorbed into the adsorbent or dessicant to form a dehydrated feed stream 130. As the adsorbent or dessicant becomes saturated with water, acetone, and other contaminants, the molecular adsorbent or dessicant loses activity and forms spent adsorbent or dessicant. Preferably, the dehydrated feed stream 130 includes water that is present in an amount of about 1 wt. ppm or less, and acetone that is present in an amount of about 20 wt. ppm or less.

In the regenerative mode, the second dryer 122, which was previously in the dehydration mode as discussed in the foregoing paragraph with respect to the first dryer 120, contains spent adsorbent or dessicant. A portion 134 of the dehydrated feed stream 130 is passed through a heater 136 and is heated to a temperature preferably of from about 200 to about 320° C. to form a heated product regeneration stream 138. In an exemplary embodiment, the heated product regeneration stream 138 is introduced to the second dryer 122 that is operating at regenerative conditions including a temperature of from about 200 to about 320° C. The heated product regeneration stream 138 contacts the spent adsorbent or dessicant and removes water, acetone, and other contaminants to restore activity to the molecular sieve, forming regenerated adsorbent or dessicant and a spent product regeneration stream 140. The spent product regeneration stream 140 is passed through a cooler 142 where it is cooled to a temperature of about 60° C. or less, and also optionally through a further coalescer 145, and is combined with the hydrocarbon-containing feed stream 101, as shown in FIG. 1. The non-recycled portion of stream 130 (shown as portion 135) is sent downstream as a decontaminated product for use in additional hydrocarbon processing systems, such as an alkylation system.

Accordingly, embodiments of the present disclosure employ a novel combination of a water wash and molecular sieve adsorbers that are used to remove acetone from an olefinic feedstock more efficiently than either a water wash or molecular sieve adsorbers alone. The use of a water wash upstream of the molecular sieve adsorbers allows the use of the dried olefin feed as regenerant and the spent regenerant can be recirculated upstream of the water wash. The use of a water wash together with molecular sieve adsorbers can allow for the use of less water overall, such as less than the current typical acetone wash rates.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the application in any way. Rather, the foregoing detailed description will provide those skilled in the art with a convenient road map for implementing one or more embodiments, it being understood that various changes may be made in the function and arrangement of elements described in an exemplary embodiment without departing from the scope, as set forth in the appended claims.

What is claimed is:

1. A method for removing acetone from a hydrocarbon stream comprising the steps of:
mixing an olefin-containing hydrocarbon stream and a wash water stream to form a mixed olefin/wash water stream, wherein the olefin-containing hydrocarbon stream comprises acetone impurities, and wherein mixing comprises dissolving at least a portion of the acetone impurities in to the wash water stream;
coalescing the mixed olefin/water stream to separate the wash water stream with acetone dissolved therein from the hydrocarbon stream, wherein the separated hydrocarbon stream comprises non-coalesced wash water and acetone;
drying the separated hydrocarbon stream to remove at least a portion of the non-coalesced wash water and acetone therefrom and to form a dried hydrocarbon product stream;
recycling a portion of the dried hydrocarbon product stream to form a product regeneration stream; and
mixing the product regeneration stream with at least a portion of the olefin-containing hydrocarbon stream and the wash water stream.

2. The method of claim 1, wherein mixing comprises mixing the hydrocarbon stream and the wash water at a ratio of water to hydrocarbons of less than 0.3:1, by volume.

3. The method of claim 1, wherein coalescing comprises coalescing in a packed-bed coalescer.

4. The method of claim 1, further comprising recycling the separated wash water stream.

5. The method of claim 1, further comprising passing the separated wash water stream to an alkylate wash tower of a downstream hydrocarbon alkylation system.

6. The method of claim 1, wherein drying comprises drying in a lead/lag dehydration unit.

7. The method of claim 6, wherein drying comprises drying in a lead dryer unit comprising an adsorbent or dessicant.

8. The method of claim 7, further comprising regenerating spent adsorbent or dessicant in a lag dryer unit.

9. The method of claim 8, wherein recycling the portion of the dried hydrocarbon product stream comprises recycling to the lag dryer unit.

10. The method of claim 1, further comprising passing the dried hydrocarbon product stream to a hydrocarbon alkylation system.

11. A system for removing acetone from a hydrocarbon stream comprising:
a mixing unit that mixes an olefin-containing hydrocarbon stream and a wash water stream to form a mixed olefin/wash water stream, wherein the olefin-containing hydrocarbon stream comprises acetone impurities, and wherein mixing comprises dissolving at least a portion of the acetone impurities in to the wash water stream;
a coalescer unit that coalesces the mixed olefin/water stream to separate the wash water stream with acetone dissolved therein from the hydrocarbon stream, wherein the separated hydrocarbon stream comprises non-coalesced wash water and acetone; and
a dehydration unit that dries the separated hydrocarbon stream to remove at least a portion of the non-coalesced wash water and acetone therefrom and to form a dried hydrocarbon product stream, wherein the dehydration unit employs a recycle portion of the dried hydrocarbon product as a product regeneration stream.

12. The system of claim 11, wherein the mixing unit comprises an in-line mixer, a scrubber, or a counter-current tower.

13. The system of claim 11, wherein the coalescer unit comprises a packed-bed coalescer.

14. The system of claim 11, wherein the dehydration unit comprises a lead/lag dryer system.

15. The system of claim 14, wherein the lead/lag dryer system comprises an adsorbent or dessicant.

16. The system of claim 15, wherein a lead dryer of the lead/lag dryer system produces the dried hydrocarbon product.

17. The system of claim 16, wherein a lag dryer of the lead/lag dryer system receives the product regeneration stream.

18. The system of claim 17, wherein the product regeneration stream regenerates the adsorbent or dessicant within the lag dryer.

19. A method for removing acetone from an olefin-containing hydrocarbon stream comprising the steps of:
mixing an olefin-containing hydrocarbon stream and a wash water stream to form a mixed olefin/wash water stream, wherein the olefin-containing hydrocarbon stream comprises acetone impurities, and wherein mixing comprises dissolving at least a portion of the acetone impurities in to the wash water stream;
coalescing the mixed olefin/water stream to separate the wash water stream with acetone dissolved therein from the hydrocarbon stream, wherein the separated hydrocarbon stream comprises non-coalesced wash water and acetone;
drying the separated hydrocarbon stream in a lead/lag dryer system comprising a lead dryer and a lag dryer to remove at least a portion of the non-coalesced wash water therefrom and to form a dried hydrocarbon product stream, wherein the dried hydrocarbon product stream is formed in a lead dryer of the lead/lag dryer system;
recycling a portion of the dried hydrocarbon product stream to form a product regeneration stream;
contacting the product regeneration stream with the lag dryer of the lead/lag dyrer system; and
after contacting, mixing the product regeneration stream with at least a portion of the olefin-containing hydrocarbon stream and the wash water stream.

20. The method of claim 19, wherein contacting comprises regenerating an adsorbent or dessicant with the lag dryer of the lead lag dryer system.
